(19) United States
(12) Patent Application Publication
(10) Pub. No.: US 2011/0021634 A1
(43) Pub. Date: Jan. 27, 2011

(54) PROCESSES FOR PREPARING METFORMIN HYDROCHLORIDE

(76) Inventors: Pranav Dushyant PATEL, Gujarat (IN); Dinesh Ramanlal Patel, Gujarat (IN)

Correspondence Address: LADAS & PARRY LLP, 26 WEST 61ST STREET, NEW YORK, NY, 10023, US

(21) Appl. No.: 12/817,629

(22) Filed: Jun. 17, 2010

(30) Foreign Application Priority Data
Jun. 18, 2009 (IN) 1459/MUM/2009

(51) Int. Cl. A61K 31/155; C07C 277/08; C07C 279/26; A61P 3/10

(52) U.S. Cl. 514/635; 564/233

(57) ABSTRACT

A process for reducing dimethylamine content in metformin hydrochloride is disclosed. The process comprises:
(a) providing metformin hydrochloride having dimethylamine content more than 15 ppm;
(b) pulverizing the metformin hydrochloride;
(c) slurrying the metformin hydrochloride in one or more C₁-C₄ alcohol solvents; and
(d) isolating the metformin hydrochloride.

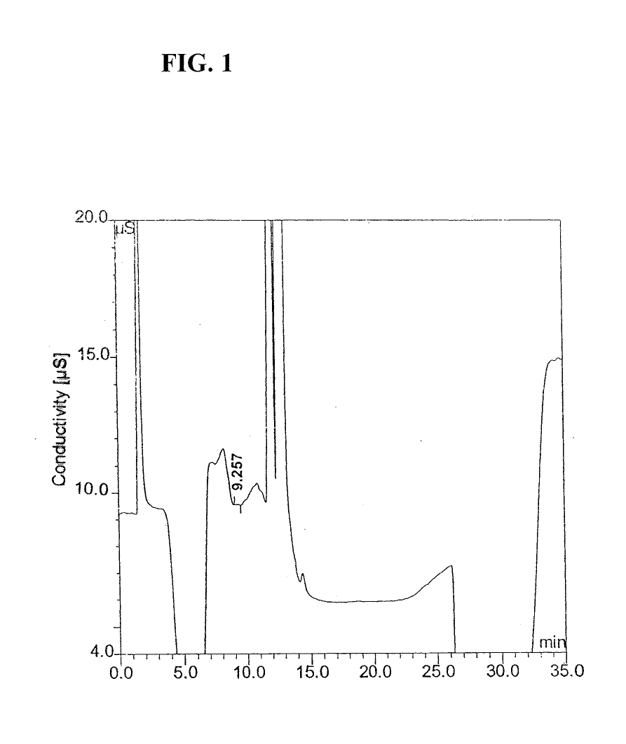

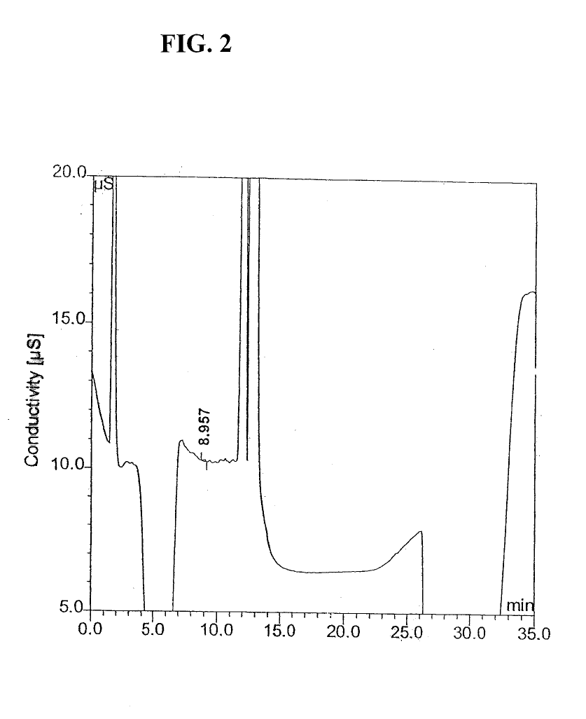

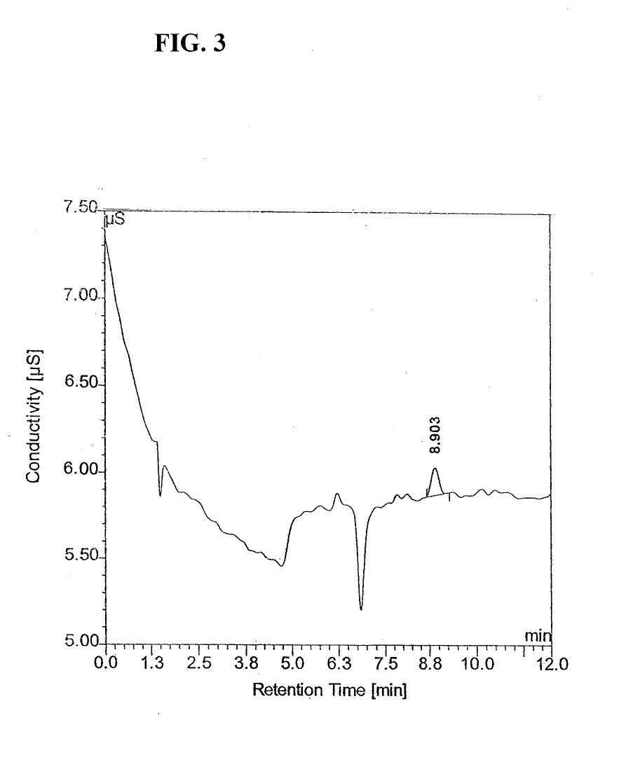

### FIELD OF THE INVENTION

The field of the invention relates to processes for the preparation of metformin hydrochloride of Formula (I). More particularly, it relates to processes for the preparation of metformin hydrochloride substantially free from dimethylamine. The invention also relates to pharmaceutical compositions that include the metformin hydrochloride substantially free from dimethylamine and use of said composition for treating diabetes.

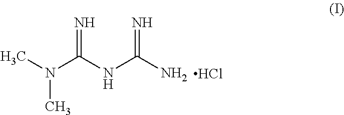

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

N,N-dimethyl imidodicarbonimidic diamide is a biguanide drug, the generic name of which is metformin. When this drug is administered to type 2 diabetic patients or glucose intolerant patients, it can exhibit blood glucose lowering action by controlling glucose formation in the liver and increasing glucose utilization in muscles and improve lipid metabolism, thus preventing the development and deterioration of diabetes complications and treating diabetes complications.

It can be seen in several papers that only metformin among oral anti-diabetic drugs is a first-choice drug. Particularly, it was proved that metformin has the effect of activating AMPK, and thus the propriety of clinical effects thereof was demonstrated.

It was reported that AMPK is a key enzyme physiologically controlling metabolism of carbohydrate and lipid, and metformin is effective in normalizing high glucose level, improving the condition of lipid, normalizing amenorrhea, ovulation and pregnancy, treating fatty liver, and preventing and treating p53 gene-deficient cancers by activating said enzyme.

According to a report by the Abramson Cancer Center of the University of Pennsylvania, metformin, an AMPK activator, is effective for the prevention and treatment of p53 gene-deficient cancers [Monica Buzzai, et al. Systemic Treatment with the Antidiabetic Drug Metformin Selectively Impairs p53 gene-Deficient Tumor Cellgrowth, Cancer Res 2007; 67:(14); Jul. 15, 2007].

The free base form of metformin is pharmaceutically useful, but has low stability. For this reason, metformin is administered in the form of a pharmaceutically acceptable acid addition salt. Several acid addition salts other than metformin hydrochloride are known for example, in U.S. Pat. Nos. 4,028,402; 4,835,184; 3,903,141; 3,957,853; 3,651,132; 6,031,004; CN 196266IA; International (PCT) Publication Nos. WO 2005/033067; 2008/093984; 2008/061456; 2009/038396; 2005/033067; KR 2009/005513A; FR 2 796 551; and 2 796 940.

Several processes have been reported for the preparation of metformin hydrochloride for example, in FR 2 322 860 B1; CN100391939 C; IN 189077 A1; and WO 2005/033089.

The processes reported in the art for the preparation of metformin and its pharmaceutically acceptable salts does not disclose any limit for the content of dimethylamine in the finished Active Pharmaceutical Ingredient (API). As per the literature reference of Clinica Chimica Acta, 233 (1995) 81-88 which discloses that dimethylamine has been suspected as a possible nerotoxin in uraemic patients where it is sequestered intracellularly and occurs in higher than normal concentrations in the intestine, blood, cerebrospinal fluid and brain tissues. These high concentrations apparently correlating with impaired neuropsychological function'.

Moreover, the reference cited by the United States Department of Labor under the site of www.osha.gov which clearly discloses the exposure limit of dimethylamine as stated below:

‘OSHA PEL [Occupational Safety and Health Administration (OSHA), Permissible Exposure Limit (PEL)] for dimethyamine is 10 ppm 918 mg per cubic meter as an 8-hour time-weighted average (TWA) concentration [29 CFR 1910.1000]
‘National Institute for Occupational Safety and Health (NIOSH) has established a recommended exposure limit (REL) for dimethylamine of 10 ppm (18 mg per cubic meter as a time-weighted average (TWA) concentration for upto 10-hour workday and a 40-hour workweek [NIOSH 1992].
‘American Conference of Governmental Industrial Hygienists (ACGIH) has assigned dimethylamine a short-term exposure limit (STEL) of 15 ppm (27.6 mg per cubic meter) for periods not to exceed 15 minutes [ACGIH 1994, p. 19]

The control of dimethylamine content below the level of 15 ppm can be done only through in-process scavenging of dimethylamine in acidic conditions, i.e., either in work-up of main reaction or during purification.

There are numerous methods available in the prior art for the removal of dimethylamine using scavengers or biofiltration techniques etc. J. Chem. Soc., Perkin Trans, 1, 2000, 3815-4195 discloses on page no. 4143 the use of chloroformyl polystyrene resin for the scavenging of dimethylamine. [Synlett, 2000, 205] and use of Amberlyst A-15 Proton form for scavenging amines for purification purpose [Tetrahedron Lett., 1997, 38, 197]

Chemosphere, Vol. 72 (2), 2008, pages 250-256 discloses a biofiltration technique for reducing the level of trimethylamine, dimethylamine and methylamine by immobilized Paracoccus sp. CP2 and Arthrobacter sp. CP1.

The references cited in the above art for reducing the level of dimethyamine do not disclose the process for the removal of dimethylamine in metformin hydrochloride. Further, these references disclose the approaches like use of resins or biofiltration techniques, which may not be suitable for large-scale production. Thus, in order to reduce the dimethylamine content below the permissible limit of 15 ppm in metformin hydrochloride, there is still need of an improved process for preparing metformin hydrochloride that provides reduced level of dimethylamine.

### SUMMARY OF THE INVENTION

In one general aspect there is provided a process for reducing dimethylamine content in metformin hydrochloride. The process includes providing metformin hydrochloride having dimethylamine content more than 15 ppm; pulverizing the metformin hydrochloride; slurrying the metformin hydrochloride in one or more C₁-C₄ alcohol solvents; and isolating the metformin hydrochloride. The steps of pulverization and slurrying can be performed in any sequential order.

The process may include further drying of the product obtained.

The process may produce the metformin hydrochloride containing dimethylamine content less than 10 ppm. In particular, it may produce the metformin hydrochloride containing dimethylamine content less than 5 ppm.

In another general aspect there is provided a process for the purification of metformin hydrochloride. The process includes obtaining a solution of metformin hydrochloride in water and recovering the metformin hydrochloride by removal of the water.

Removing the water may include, for example, one or more of evaporation, distillation and distillation under vacuum. The process may include further forming of the product so obtained into a finished dosage form.

The process may produce the metformin hydrochloride having dimethylamine content less than the starting metformin hydrochloride.

In another general aspect there is provided a process for the preparation of metformin hydrochloride substantially free from dimethylamine. The process includes obtaining a solution of metformin hydrochloride in water; optionally clarifying the solution by treating with charcoal; removing the water to obtain a residue; treating the residue with one or more C₁-C₄ alcohols to obtain slurry; pulverizing the slurry; and isolating the metformin hydrochloride substantially free from dimethylamine.

In another aspect there is provided a process for the preparation of metformin hydrochloride of Formula (I)

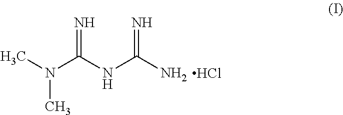

substantially free from dimethylamine. The process includes:
(a) reacting dicyanodiamide of Formula (II)

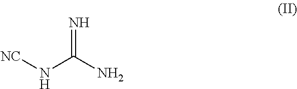

with dimethylamine hydrochloride of Formula (III)

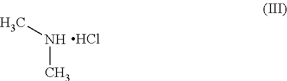

in one or more hydrocarbon solvents;
(a) extracting reaction mass with water to obtain a solution;
(b) optionally treating the solution with charcoal;
(c) removing water to obtain a residue;
(d) treating the residue with one or more C₁-C₄ alcohols to obtain a slurry;
(e) pulverizing the slurry; and
(f) isolating the metformin hydrochloride substantially free of dimethylamine.

In another aspect there is provided metformin hydrochloride substantially free from dimethylamine.

In another general aspect there is provided a pharmaceutical composition comprising a therapeutically effective amount of metformin hydrochloride substantially free from dimethylamine, and one or more pharmaceutically acceptable carriers, excipients or diluents.

In another general aspect there is provided a method of treating diabetes in a warm-blooded animal, the method comprising providing a dosage form to the warm-blooded animal that includes metformin hydrochloride substantially free from dimethylamine.

The details of one or more embodiments of the inventions are set forth in the description below. Other features, objects and advantages of the inventions will be apparent from the description.

### BRIEF DESCRIPTION OF DRAWINGS

FIG. I: Chromatogram for metformin hydrochloride for determination of dimethylamine as per Example-1.

FIG. II: Chromatogram for metformin hydrochloride for determination of dimethylamine as per Example-2.

FIG. III: Chromatogram for metformin hydrochloride standard for determination of dimethylamine.

### DETAILED DESCRIPTION OF THE INVENTION

The term “substantially free” from dimethylamine as used herein refers to metformin hydrochloride containing less than about 10 ppm of dimethylamine, for example less than about 5 ppm of dimethylamine when measured by Ion chromatography method.

The term “pulverizing” as used herein refers to a technique or a method by which particle size can be reduced and includes techniques such as milling, grinding, micronizing, and the like.

The inventors have developed a process for reducing dimethylamine content in metformin hydrochloride. The process includes the step of pulverizing metformin hydrochloride having dimethylamine content more than 15 ppm; slurrying the metformin hydrochloride in one or more C₁-C₄ alcohol solvents; and isolating the metformin hydrochloride. The pulverization and slurrying steps can be performed in any sequential order.

The pulverizing of metformin hydrochloride can be done by milling or grinding or micronizing metformin hydrochloride to very fine particles.

The slurrying of metformin hydrochloride can be done in one or more C₁-C₄ alcohol solvents. A suitable alcohol includes one or more of methanol, ethanol, propanol, isopropanol, butanol and isobutaneol.

In another aspect there is provided a process for the purification of metformin hydrochloride. The process includes obtaining a solution of metformin hydrochloride in water and recovering the metformin hydrochloride by removal of the water. The solution of water may be prepared under nitrogen atmosphere and may be heated at a temperature from about ambient temperature to about reflux temperature. In particular, the solution may be prepared at about 80° C. to about 95° C.

The process may produce metformin hydrochloride having reduced dimethylamine content than the starting metformin hydrochloride.

In another aspect there is provided a process for the preparation of metformin hydrochloride substantially free from dimethylamine. The process comprising:

(a) obtaining a solution of metformin hydrochloride in water;
(b) optionally clarifying the solution by treating with charcoal;
(c) removing the water to obtain a residue;
(d) treating the residue with one or more C₁-C₄ alcohols to obtain a slurry;
(e) pulverizing the slurry; and
(f) isolating the metformin hydrochloride substantially free from dimethylamine.

In general, the solution of water may be prepared under nitrogen atmosphere and may be heated at a temperature from about ambient temperature to about reflux temperature. In particular, the solution may be prepared at about 80° C. to about 95° C. The nitrogen gas may be purged for about 15 minutes to 1 hour. In particular, it may be purged for about 30 minutes. Alternatively, such a solution may be obtained directly from a reaction in which metformin hydrochloride is formed. The solution may be heated under nitrogen atmosphere.

The solution can be optionally clarified to remove colored and other suspended matters by treatment with charcoal.

The water may be removed from the solution by a technique which includes, for example, distillation, distillation under vacuum, and evaporation. The residue obtained may be treated with one or more of C₁-C₄ alcohols to obtain slurry. A suitable alcohol includes one or more of methanol, ethanol, propanol, isopropanol, butanol and isobutanol.

The process may produce metformin hydrochloride which is substantially free from dimethylamine.

In another aspect there is provided a process for the preparation of metformin hydrochloride substantially free from dimethylamine. The process includes:

(a) reacting dicyanodiamide of Formula (II)

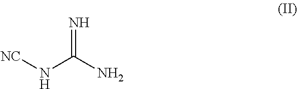

with dimethylamine hydrochloride of Formula (III)

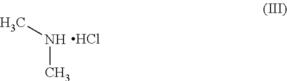

in one or more hydrocarbon solvents;

(b) extracting reaction mass with water to obtain a solution;
(c) optionally treating the solution with charcoal;
(d) removing water to obtain a residue;
(e) treating the residue with one or more C₁-C₄ alcohols to obtain a slurry;
(f) pulverizing the slurry; and
(g) isolating the metformin hydrochloride substantially free from dimethylamine.

The reaction of dicyanodiamide of Formula (II) with dimethylamine hydrochloride of Formula (III) may be carried out in a hydrocarbon solvent. A suitable hydrocarbon solvent includes one or more of toluene, xylene, ethylbenzene, cyclohexane, hexane, cyclopentane, pentane, and heptanes. In particular, toluene and xylene may be used. The reaction mixture may be heated at a temperature from about 50° C. to about 150° C.

After the completion of the reaction, the reaction mass may be extracted with water at ambient temperature to reflux temperature, for example, at about 80° C. to about 95° C. The solution may be purged with nitrogen gas for about 15 minutes to one hour.

The solution can be optionally clarified to remove colored and other suspended matters by treatment with charcoal.

The water may be removed from the solution by a technique which includes, for example, distillation, distillation under vacuum, and evaporation. The residue obtained may be treated with one or more of C₁-C₄ alcohols to obtain slurry. A suitable alcohol includes one or more of methanol, ethanol, propanol, isopropanol, butanol and isobutanol.

The present invention is further illustrated by the following example which is provided merely to be exemplary of the invention and do not limit the scope of the invention. Certain modifications and equivalents will be apparent to those skilled in the art and are intended to be included within the scope of the present invention.

### Example-1

### Preparation of Metformin Hydrochloride (I)

Xylene (400 mL) and dicyanodiamide (100 g) were taken in a round-bottom flask. The reaction mixture was heated at 80° C. Dimethylamine hydrochloride (117 g) was added portion-wise within 2 hours. The reaction mass was stirred for 3 hours. The reaction mixture was further heated to 100° to 105° C. followed by heating to 140° C. The reaction mass was stirred for 4 hours and cooled to 95° C. The reaction mass was treated with water (200 mL) and layers were separated. The organic layer was again extracted with water (50 mL). The combined aqueous layer was treated with charcoal (3 g) and stirred for 20 minutes. The reaction mass was filtered through a hyflowbed and washed with water (50 mL). The filtrate was taken in a round-bottom flask at 50° C. and N₂ gas was purged for 30 minutes. The filtrate was distilled to remove water completely under vacuum at 65° C. The residue thus obtained was treated with methanol (110 mL) at 40° C. to 45° C. and cooled to 20° C. to 25° C. The product was filtered and washed with chilled methanol (50 mL). The wet-cake thus obtained was treated with water at 50° C. along with N₂ gas purging for 30 minutes. The solution was distilled to remove water completely under vacuum at 65° C. The residue thus obtained was treated with methanol (80 mL) at 40° C. to 45° C. to prepare the slurry. The slurry was pulverized under high-speed grinder for wet grinding for 25 minutes. The reaction mass was filtered and dried. The wet-cake was washed with chilled methanol (30 mL). The product was dried at 65° C. to 70° C. to obtain 160 g metformin hydrochloride having dimethylamine content less than 5 ppm.

### Example-2

### Preparation of Metformin Hydrochloride (I)

Toluene (500 mL) and dicyanodiamide (100 g) were taken in a round-bottom flask at 25° C. to 35° C. The reaction mixture was heated at 80° C. Dimethylamine hydrochloride (117 g) was added portion-wise within 2 hours. The reaction mass was stirred for 3 hours. The reaction mixture was further heated to 100° to 105° C. followed by heating to reflux temperature. The reaction mass was stirred for 4 hours and cooled to 95° C. The reaction mass was treated with water (200 mL) and layers were separated. The organic layer was again extracted with water (50 mL). The combined aqueous layer was treated with charcoal (3 g) and stirred for 20 minutes. The reaction mass was filtered through hyflowbed and washed with water (50 mL). The filtrate was taken in a round-bottom flask at 50° C. and N₂ gas was purged for 30 minutes. The filtrate was distilled to remove water completely under vacuum at 65° C. The residue thus obtained was treated with methanol (110 mL) at 40° C. to 45° C. and cooled to 20° C. to 25° C. The product was filtered and washed with chilled methanol (50 mL). The wet-cake thus obtained was treated with water at 50° C. along with N₂ gas purging for 30 minutes. The solution was distilled to remove water completely under vacuum at 65° C. The residue thus obtained was treated with methanol (80 mL) at 40° C. to 45° C. to prepare the slurry. The slurry was pulverized under high-speed grinder for wet grinding for 25 minutes. The reaction mass was filtered and dried. The wet-cake was washed with chilled methanol (30 mL). The product was dried at 65° C. to 70° C. to obtain 155 g metformin hydrochloride having dimethylamine content less than 5 ppm.

### Example-3

### Estimation of Dimethylamine in Metformin Hydrochloride by Ion Chromatography

### Chromatographic Conditions:

### Column: IoncPac CG17 (Guard Column)+

IoncPac CS 17 (Analytical Column
   Eluent A: 50 mM Methanesulphonic acid
   Eluent B: High purity water

### Eluent C: 90% Acetonitrile

Detector: Conductivity detector
Suppressor: CSRS-3000, in external water mode
SRS current: 150 mA
Injection volume: For Exercise I: 20 μl
For Exercise II: 100 μl.
   Diluent: 2 mM Methanesulphonic acid

### Program for Standard:

| Time (min) | Flow rate: ml/min | % A | % B | % C |
|---|---|---|---|---|
| 0.00 | 1.2 | 4 | 96 | 0 |
| 12.00 | 1.2 | 4 | 96 | 0 |

### Program for Sample:

| Time (min) | Flow rate: ml/min | % A | % B | % C |
|---|---|---|---|---|
| 0.00 | 1.2 | 4 | 96 | 0 |
| 10.00 | 1.2 | 4 | 96 | 0 |
| 10.10 | 1.5 | 60 | 38 | 2 |
| 24.00 | 1.5 | 60 | 38 | 2 |
| 21.10 | 1.2 | 4 | 96 | 0 |
| 35.00 | 1.2 | 4 | 96 | 0 |

### Preparation of Standard:

### Primary Standard Stock:

Weigh accurately about 45 mg of dimethylamine hydrochloride in a 100 ml volumetric flask. Dissolve and dilute upto the mark with diluent and Mix.

### Intermediate Standard Stock A:

Dilute 2 ml of primary standard stock solution to 50 ml with diluent and mix.

### Intermediate Standard Stock B:

Dilute 10 ml of the intermediate standard stock solution A to 100 ml with diluent.

### Standard Solution:

Dilute 7.5 ml of the intermediate standard stock solution B to 100 ml with diluent.

### Sample Preparation:

Transfer an accurately weighed quantity of about 50 mg of metformin hydrochloride sample to a 10 ml volumetric flask, dissolve and dilute to volume with diluent and mix.

### Calculations:

Dimethylamine Content (ppm):

$$= \frac{AT}{AS} \times \frac{WS}{DS} \times \frac{DT}{WT} \times \frac{P}{100} \times \frac{45.08}{81.58} \times 1000$$

AT=Area of Dimethylamine in sample
AS=Average Area of Dimethylamine in standard
WS=Weight of the Dimethylamine hydrochloride in mg
WT=Weight of the sample in g
DS=Dilution of standard
DT=Dilution of sample
45.08=Molecular weight of the Dimethylamine
81.58=Molecular weight of the Dimethylamine Hydrochloride
Results are Expressed in Terms of “ppm”

While the present invention has been described in terms of its specific embodiments, certain modifications and equivalents will be apparent to those skilled in the art and are intended to be included within the scope of the present invention.

1. A process for reducing dimethylamine content in metformin hydrochloride, the process comprising the steps of:
(a) providing metformin hydrochloride having dimethylamine content more than 15 ppm;
(b) pulverizing the metformin hydrochloride;
(c) slurrying the metformin hydrochloride in one or more C₁-C₄ alcohol solvents; and
(d) isolating the metformin hydrochloride
wherein steps (b) and (c) may be performed in any sequential order.

2. The process as claimed in claim 1, wherein the isolated metformin hydrochloride contains dimethylamine less than 10 ppm.

3. The process as claimed in claim 1, wherein the isolated metformin hydrochloride contains dimethylamine less than 5 ppm.

4. The process as claimed in claim 1, wherein the alcohol comprises one or more of methanol, ethanol, propanol, isopropanol, butanol and isobutanol.

5. The process according to claim 1, wherein before step (c) for the purification of metformin hydrochloride, the process comprising obtaining a solution of water is added to the metformin hydrochloride to form a solution in water and recovering the metformin hydrochloride is recovered by removal of the water.

6. The process as claimed in claim 5, wherein the solution is prepared under nitrogen atmosphere.

7. The process as claimed in claim 5, wherein the solution is prepared at a temperature from about ambient to about reflux temperature.

8. The process as claimed in claim 7, wherein the temperature is from about 80° C. to about 95° C.

9. (canceled)

10. A process for the preparation of metformin hydrochloride substantially free from dimethylamine, the process comprising the steps of:
(a) obtaining a solution of metformin hydrochloride in water;
(b) optionally clarifying the solution by treating with charcoal;
(c) removing the water to obtain a residue;
(d) treating the residue with one or more C₁-C₄ alcohols to obtain a slurry;
(e) pulverizing the slurry; and
(f) isolating the metformin hydrochloride substantially free from dimethylamine.

11. The process as claimed in claim 10, wherein the solution is prepared under nitrogen atmosphere.

12. The process as claimed in claim 10, wherein the solution of metformin hydrochloride is prepared at a temperature from about ambient to about reflux temperature.

13. The process as claimed in claim 12, wherein the temperature is from about 80° C. to about 95° C.

14. The process as claimed in claim 10, wherein the removal of water is carried out by one or more of evaporation, distillation or distillation under vacuum.

15. The process as claimed in claim 10, wherein the alcohol comprises one or more of methanol, ethanol, propanol, isopropanol, butanol and isobutanol.

16. A process for the preparation of metformin hydrochloride of Formula (I) 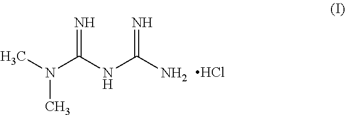
substantially free from dimethylamine, the process comprising the steps of:
(a) reacting dicyanodiamide of Formula (II) 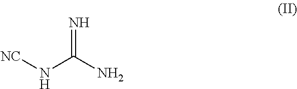
with dimethylamine hydrochloride of Formula (III) 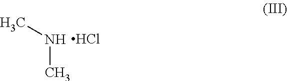
in one or more hydrocarbon solvents;
(b) extracting reaction mass with water to obtain a solution;
(c) optionally treating the solution with charcoal;
(d) removing water to obtain a residue;
(e) treating the residue with one or more C₁-C₄ alcohols to obtain a slurry;
(f) pulverizing the slurry; and
(g) isolating the metformin hydrochloride substantially free from dimethylamine.

17. The process as claimed in claim 16, wherein the hydrocarbon solvent comprises one or more of toluene, xylene, ethylbenzene, cyclohexane, hexane, cyclopentane, pentane, and heptane.

18. The process as claimed in claim 17, wherein the solvent is toluene or xylene.

19. The process as claimed in claim 16, wherein step (a) is carried out at a temperature from about 50° C. to about 150° C.

20. The process as claimed in claim 16, wherein the solution is prepared under nitrogen atmosphere.

21. The process as claimed in claim 16, wherein the removal of water is carried out by one or more of evaporation, distillation or distillation under vacuum.

22. The process as claimed in claim 16, wherein the alcohol comprises one or more of methanol, ethanol, propanol, isopropanol, butanol and isobutanol.

23. The process as claimed in claim 16, wherein the pulverization is carried out by one or more of milling, grinding, and micronizing.

24. Metformin hydrochloride substantially free from dimethylamine.

25. Metformin hydrochloride according to claim 24, containing dimethylamine less than 5 ppm.

26. A pharmaceutical composition comprising a therapeutically effective amount of metformin hydrochloride substantially free from dimethylamine, and one or more pharmaceutically acceptable carriers, excipients or diluents.

27. A method of treating diabetes in a warm-blooded animal, the method comprising providing a dosage form to the warm-blooded animal that includes metformin hydrochloride substantially free from dimethylamine.

28. (canceled)
